(19) United States
(12) Patent Application Publication
(10) Pub. No.: US 2018/0163177 A1
(43) Pub. Date: Jun. 14, 2018

(54) SERUM-FREE CULTURE MEDIUM AND METHOD FOR EXPANDING HEMATOPOIETIC STEM CELLS

(71) Applicant: HealthBanks Biotech Co. Ltd., Taipei (TW)

(72) Inventors: Wei-Yu Lo, Taipei (TW); Pei-Chi Tseng, Taipei (TW); Chao-Hsun Yu, Taipei (TW)

(73) Assignee: HealthBanks Biotech Co. Ltd., Taipei (TW)

(21) Appl. No.: 15/832,774

(22) Filed: Dec. 6, 2017

Related U.S. Application Data
(60) Provisional application No. 62/432,566, filed on Dec. 11, 2016.

(51) Int. Cl. C12N 5/0789

(52) U.S. Cl. CPC C12N 5/0647; C12N 2500/90; C12N 2501/125; C12N 2501/145; C12N 2501/26; C12N 2501/22; C12N 2500/38; C12N 2501/392; C12N 2501/2303; C12N 2501/2306; C12N 2502/1388

(57) ABSTRACT

A serum-free culture medium for hematopoietic stem cell (HSC) expansion is provided. The serum-free culture medium includes a serum-free base medium, cytokines, an umbilical cord mesenchymal stem cell conditioned medium and supplemental components. The cytokines comprise stem cell factor, thrombopoietin and hematopoietic growth factor Flt3 ligand. The umbilical cord mesenchymal stern cell conditioned medium is derived from culturing human umbilical cord mesenchymal stem cells. The supplemental components comprise vitamin C, vitamin E or a combination of vitamin C and vitamin E.

### CROSS-REFERENCE TO RELATED APPLICATION

This application claims the priority benefit of U.S. provisional application Ser. No. 62/432,566, filed on Dec. 11, 2016. The entirety of the above-mentioned patent application is hereby incorporated by reference herein and made a part of this specification.

### BACKGROUND OF THE INVENTION

### 1. Field of the Invention

The present invention generally relates to a serum-free culture medium, in particular, relates to a serum-free culture medium and a method for expanding hematopoietic stem cells using such serum-free culture medium.

### 2. Description of Related Art

Umbilical cord blood transplantation (UCBT) is a new therapy for patients making it possible to treat previously incurable diseases. However, UCBT in adults is limited by the small number of primitive hematopoietic stem cells (HSC) available in each graft. The small number of primitive HSC results in delayed engraftment after transplantation. Efforts to expand umbilical cord blood (UCB) progenitors ex vivo have not been very successful. The ex vivo expansion often esults in the expansion of mature HSC, instead of immature HSC. In addition, ex vivo expansion of UCB HSC may result in defects that can promote apoptosis, disrupt marrow homing, and initiate cell cycling etc.

The difficulties in ex vivo expansion of HSC arise from the requirements for various factors for the growth and proliferation of the primitive HSC. Earlier studies show that the ex vivo growth of hematopoietic stem cells requires cytokines and hematopoietic growth factors produced by other tissues present in the serum. These factors, for example, include erythropoietin, interleukin-3 (IL-3), granulocyte macrophage-colony stimulating factor (GM-CSF), granulocyte-colony stimulating factor (G-CSF), stem cell factor (SCF), interleukin-11 (IL-11), etc.

Due to the requirements for complex factors, it has been difficult to generate sufficient HSC numbers and to avoid differentiation of the starting cell population. From in vitro studies, it has been found that controls of HSC self-renewal and differentiation in cell cultures are difficult. Protocols that are based on hematopoietic cytokines have failed to support reliable amplification of immature stern cells in culture, suggesting that additional factors (other than cytokines) are also required.

Most primitive hematopoietic stem cells typically have CD34 on their cell membranes. CD34 is a surface glycoprotein of unknown function. Cells that bear the CD34 antigen are thought to be responsible for multi-lineage engraftment. While CD34 is present on most proliferative cells, its appearance on other cells is rare—e.g., found on approximately 1% of collected mononuclear cells (MNCs). Since proliferative hematopoietic stem cells are CD34⁺ cells, hematopoietic expansion starting with CD34⁺ cells have greater potential. However, starting with CD34⁺ cells alone would not be successful due to the lack of accessory cells that may provide cytokines and other stimulatory factors. Thus, hematopoietic stem cell expansion is often carried out in the presence of serum and other tissue as feeder layers.

The requirement for serum is undesirable due to possible contaminations and adverse immune responses. Therefore, there have been efforts to find serum-free substitutes. For example, U.S. Pat. No. 5,405,772 discloses a serum-free or serum-depleted medium for culturing hematopoietic stem cells and bone marrow stromal cells, and U.S. Pat. No. 6,733,746 discloses a serum-free medium for expansion of CD34⁺ hematopoietic stem cells and cells of myeloid lineage.

While these prior art efforts have provided useful media for expansion of hematopoietic stem cells, there is still a need for better media and methods for the expansion of hematopoietic stem cells.

### SUMMARY OF THE INVENTION

Accordingly, the present invention is directed to a serum-free culture medium that can be used for expanding hematopoietic stem cells.

In one embodiment of the invention, a serum-free culture medium for hematopoietic stem cell (HSC) expansion is provided. The serum-free culture medium includes a serum-free base medium, cytokines, an umbilical cord mesenchymal stern cell conditioned medium and supplemental components. The cytokines comprise stern cell factor (SCF), thrombopoietin (TPO) and hematopoietic growth factor Flt3 ligand (F1t3L). The umbilical cord mesenchymal stern cell conditioned medium is derived from culturing human umbilical cord mesenchymal stern cells. The supplemental components comprise vitamin C, vitamin E or a combination of vitamin C and vitamin E.

In accordance with some embodiments of the invention, the serum-free base medium may be any serum-free medium suitable for cell cultures. Many such suitable media are known in the art. For example, U.S. Pat. No. 5,405,772 discloses a serum-free or serum-depleted medium for culturing hematopoietic stem cells and bone marrow stromal cells. U.S. Pat. No. 6,733,746 discloses a serum-free medium for expansion of CD34⁺ hematopoietic stem cells and cells of myeloid lineage. U.S. Pat. No. 8,762,074 discloses a method of determining the optimal composition of a serum-free, eukaryotic cell culture medium supplements. The disclosures of these patents are incorporated by reference in their entirety. The based media disclosed in these prior art references may be used with embodiments of the invention.

In accordance with some embodiments of the invention, the supplemental components include vitamin C.

In accordance with some embodiments of the invention, the supplemental components include vitamin E.

In accordance with some embodiments of the invention, the supplemental components comprise vitamin C and vitamin E.

In accordance with some embodiments of the invention, the supplemental components further comprise estradiol (E2).

In accordance with some embodiments of the invention, the umbilical cord mesenchymal stern cell conditioned medium is produced by a method comprising the following steps: (a) culturing human umbilical cord mesenchymal stem cells in a cell culture medium; (b) isolating the cell culture medium to obtain a conditioned cell culture medium.

In accordance with some embodiments of the invention, the method for producing the umbilical cord mesenchymal stem cell conditioned medium further comprises the step (c): concentrating the conditioned cell culture medium with a 5-10 kilodaltons cut-off membrane to obtain a concentrated umbilical cord mesenchymal stem cell conditioned medium.

In accordance with some embodiments of the invention, in the step (c) the umbilical cord mesenchymal stem cell conditioned medium is 7 to 12 times concentrated.

In accordance with some embodiments of the invention, the umbilical cord mesenchymal stem cell conditioned medium is concentrated to a protein concentration of 100 mg/ml. The umbilical cord mesenchymal stem cell conditioned medium may be concentrated to a desirable protein concentration, such as from 50-200 mg/ml, preferably from 100-150 mg/ml (e.g. 100 mg/ml, 110 mg/ml, 120 mg/ml, 130 mg/ml, 140 mg/ml or 150 mg/ml).

In accordance with some embodiments of the invention, components within the umbilical cord mesenchymal stem cell conditioned medium have a molecular weight of more than 5 kilodaltons (kDa). This may be achieved, for example, by dialysis or ultrafiltration using a membrane with a molecular weight cutoff of 5 kDa.

In accordance with some embodiments of the invention, the cytokines further comprise interleukin 3 (IL-3) and interleukin 6 (IL-6).

In accordance with some embodiments of the invention, the cytokines further comprise granulocyte colony stimulating factor (G-CSF).

In accordance with some embodiments of the invention, the major composition of the serum-free base medium comprises human albumin and albumin associated proteins and peptides, insulin, salts, sugars, amino acids, vitamins, buffers containing phenol-red, L-glutamine, and β-mercaptoethanol.

In accordance with some embodiments of the invention, the serum-free base medium may be a serum-free stem cell growth medium (SCGM) or X-VIVO 15.

In another embodiment of the invention, a method for expanding hematopoietic stem cells is described. The method comprises the following steps. A serum-free culture medium is prepared. The serum-free culture medium is prepared by mixing a serum-free base medium with cytokines, an umbilical cord mesenchymal stem cell conditioned medium and supplemental components, wherein the cytokines comprises stem cell factor, tlu⁻ombopoietin and hematopoietic growth factor Flt3 ligand, the umbilical cord mesenchymal stem cell conditioned medium is derived from culturing human umbilical cord mesenchymal stem cells, and the supplemental components comprise vitamin C, vitamin E or a combination of vitamin C and vitamin E. The hematopoietic stem cells are cultured in the serum-free culture medium for a first duration.

In accordance with some embodiments of the invention, the method for expanding hematopoietic stem cells further comprises replenishing 50-80% of the serum-free culture medium after the first duration and continuing to culture for a second duration.

In accordance with some embodiments of the invention, the hematopoietic stem cells are cultured for the first duration (e.g., 1-20 days), the medium may then be replenished with 50-80% of the serum-free culture medium and continuing to culture for a second duration (e.g., 1-20 days). This replenishment and refreshing may be repeated a few times.

According to the above, since the serum-free culture medium of the invention comprises at least a serum-free base medium, cytokines, an umbilical cord mesenchymal stem cell conditioned medium and supplemental components, thus hematopoietic stern cell expansion may be enhanced.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1A and FIG. 1B shows the results of hematopoietic stem cell expansion using defined growth media in the presence of feeder layers.

FIG. 2A and FIG. 2B shows the results of hematopoietic stem cell expansion by evaluating the effects of adding four supplements to the growth media containing feeder layers.

FIG. 3A and FIG. 3B shows the results of hematopoietic stem cell expansion by evaluating the effect of replacing feeder layers with umbilical cord mesenchymal stem cell conditioned medium.

FIG. 4A and FIG. 4B shows the results of hematopoietic stem cell expansion by evaluating the effect of replacing feeder layers with concentrated umbilical cord mesenchymal stem cell conditioned medium.

FIG. 5A and FIG. 5B shows the results of hematopoietic stem cell expansion by evaluating the effects of the four supplements in concentrated umbilical cord mesenchymal stern cell conditioned medium.

FIG. 6A and FIG. 6B shows the results of hematopoietic stern cell expansion comparing colony forming unit expansion folds and cumulative CD34+ cell expansion folds.

FIG. 7A and FIG. 7B shows the results of hematopoietic stern cell expansion by evaluating the effect of combining estradiol (E2), vitamin C and vitamin E.

FIG. 8A and FIG. 8B shows the results of hematopoietic stern cell expansion by evaluating the effect of vitamin C.

FIG. 9A and FIG. 9B shows the results of hematopoietic stem cell expansion by evaluating the effect of vitamin E.

FIG. 10A and FIG. 10B shows the results of hematopoietic stem cell expansion by evaluating the effect of combining vitamin C and vitamin E.

FIG. 11A and FIG. 11B shows the results of hematopoietic stem cell expansion by evaluating the effect of culture media replenishment.

FIG. 12A and FIG. 12B shows the results of hematopoietic stern cell expansion by using different compositions of cytokines.

FIG. 13 shows the relative expansion of the CD34+ cells relative to the total cell expansion.

FIG. 14 shows the results of hematopoietic stem cell expansion by evaluating the percentage of erythroid-lineage colony forming units.

### DESCRIPTION OF THE EMBODIMENTS

Reference will now be made in detail to the present preferred embodiments of the invention, examples of which are illustrated in the accompanying drawings. Wherever possible, the same reference numbers are used in the drawings and the description to refer to the same or like parts.

Embodiments of the invention relate to a serum-free culture medium and methods for expanding hematopoietic sterns cells (HSC). In accordance with embodiments of the invention, a medium for HSC expansion does not require serum or other auxiliary tissues/cells (e.g., feeder layers). Instead, the required factors are replaced with defined components.

Embodiments of the invention are based on defined media and factors. The serum-free culture medium of the embodiment at least includes a serum-free base medium, cytokines, an umbilical cord mesenchymal stern cell conditioned medium and supplemental components. These will be described in further detail below.

### Serum-Free Base Medium

To avoid potential contamination and adverse immune responses, the base medium should be serum-free and free of other tissues or cells. In accordance with embodiments of the invention, a suitable serum-free base medium for the expansion of hematopoietic stem cells (HSC) may be based on any suitable commercially available media. For example, the following commercially available media have been tested.

X-VIVO™ 15 is a chemically defined, serum-free medium suitable for hematopoietic cell cultures and is available from Lonza (Switzerland).

Different SCGM™ (stem cell growth medium) are available for various cell types. For example, human marrow stem cell growth medium is available from Sigma-Aldrich. In the experiment examples, the serum-free SCGM is obtained from CellGenix (No. 20802-0500) and the major compositions include human albumin and albumin associated proteins and peptides (e.g. retinol-binding protein 4, alpha-2-glycoprotein 1, Transthyretin, Haptoglobin α, hornerin precursor), insulin, salts, sugars, amino acids, vitamins, buffers containing phenol-red, L-glutamine, and β-mercaptoethanol.

Iscove's Modified Dulbecco's Media (IMDM) is a highly enriched synthetic media that are well suited for rapidly proliferating, high-density cell cultures. IMDM are available from many commercial sources, such as ThermoFisher Scientific. However, IMDM is a synthetic basic cell culture medium that typically requires the addition of serum and other growth factors for cell growth. In the experiment examples, IMDM can be used as a positive control for comparison with the serum-free base medium mentioned above for evaluating cell expansion.

In accordance with embodiments of the invention, a medium for HSC expansion, for example may comprise a commercially available defined serum-free base medium (SFM), such as SCGM described above. Based on this serum-free base medium (e.g., SCGM), selected chemicals and cytokines, as well as a conditioned medium from umbilical cord mesenchymal stern cells (UC-MSC) are added for evaluation.

### Cytokines

Six different cytokines (referred as “cytokines*6” or “CTK*6”) may be used for the cell expansion experiments. The six cytokines are recombinant human stem cell factor (rh SCF), recombinant human thrombopeietin (rh TPO), recombinant human hematopoietic growth factor Fms-related tyrosine kinase 3 ligand (rh Flt3L), recombinant human interleukin 3 (rh IL-3), recombinant human interleukin 6 (rh IL-6), and recombinant human granulocyte colony stimulating factor (rh G-CSF).

In an embodiment of the invention, the concentration of rh SCF is in a range of 20-300 ng/ml, preferably 20-100 ng/ml, more preferably 20-50 ng/ml. The concentration of rh TPO is in a range of 10-100 ng/ml, preferably 20-100 ng/ml, more preferably 20-50 ng/ml. The concentration of rh Flt3L is in a range of 50-300 ng/ml, preferably 50-100 ng/ml, more preferably 50-80 ng/ml. The concentration of rh IL-3 is in a range of 1-20 ng/ml, preferably 5-15 ng/ml, more preferably 10-15 ng/ml. The concentration of rh IL-6 is in a range of 10-100 ng/ml, preferably 10-50 ng/ml, more preferably 10-30 ng/ml. The concentration of rh G-CSF is in a range of 1-100 ng/ml, preferably 1-50 ng/ml, more preferably 1-20 ng/ml.

In a preferred embodiment, the concentration of rh SCF is 20 ng/ml. The concentration of rh TPO is 20 ng/ml. The concentration of rh Flt3L is 50 ng/ml. The concentration of rh IL-3 is 10 ng/ml. The concentration of rh IL-6 is 10 ng/ml. The concentration of rh G-CSF is 1 ng/ml.

### Umbilical Cord Mesenchymal Stem Cell Conditioned Medium

In accordance with some embodiments of the invention, the umbilical cord mesenchymal stem cell conditioned medium is derived from culturing human umbilical cord mesenchymal stem cells. In one embodiment of the invention, an umbilical cord mesenchymal stern cell conditioned medium is produced by a method comprising the steps of: (a) culturing human umbilical cord mesenchymal stem cells in a serum-free cell culture medium (e.g. serum-free SCGM) for 3-5 days, and; (b) isolating the serum-free cell culture medium to obtain a serum-free umbilical cord mesenchymal stem cell conditioned medium (hereinafter referred as “SF-UCM”).

The obtained SF-UCM can be further concentrated by the following step (c): concentrating the conditioned cell culture medium (SF-UCM) with a 5-10 kilodaltons (kDa) cut-off membrane to obtain the concentrated umbilical cord mesenchymal stem cell conditioned medium (hereinafter referred as “con. SF-UCM” or “c-SF-UCM”).

In an embodiment of the invention, the step (b) comprises centrifuging the cell culture medium with UC-MSC under the condition of 500 g and 16° C. for 10 minutes, then collecting the supernatant to obtain the conditioned cell culture medium and discarding the pellet. In some other embodiments, the step (c) mentioned above is used to obtain a concentrated umbilical cord mesenchymal stem cell conditioned medium. For example, in step (c), the conditioned cell culture medium (SF-UCM) obtained in step (b) is concentrated by using a 5-10 kDa cut-off membrane (preferably a 5 kDa cut-off membrane) to obtain a 7-12 times concentrated (in volume) conditioned medium. In some embodiments, the umbilical cord mesenchymal stem cell conditioned medium is preferably 10 times concentrated (by volume) in step (c). The concentrated conditioned medium is then filtered with a 0.22 μm filter, and the filtrate is collected to obtain the desired concentrated SF-UCM (c-SF-UCM). In some embodiments, the umbilical cord mesenchymal stern cell conditioned medium is concentrated and has a protein concentration of 50-200 mg/ml, preferably 100-150 mg/ml. In a preferred embodiment, the protein concentration of c-SF-UCM is 100 mg/ml. In some other embodiments, the umbilical cord mesenchymal stem cell conditioned medium is concentrated, so as to obtain a conditioned medium comprising protein components having a molecular weight of more than 5 kDa.

In one exemplary embodiment, the list of protein components included in the concentrated umbilical cord mesenchymal stem cell conditioned medium as identified by proteomic analysis are such as HSC expansion related proteins, HSC homing related proteins, immune modulation related proteins, neuron development related proteins, metabolic process related proteins, cellular component related proteins, vesicle transport proteins, SCGM medium components and some other unannotated components. For example, the major 91 proteins identified are presented in Table 1 shown below, wherein 48 SCGM medium components and 35 unannotated components are not listed.

TABLE 1
Protein list identified by proteomic analysis in concentrated umbilical
cord mesenchymal stem cell conditioned medium.

| | |
|---|---|
| Accession number | Protein name |

HSC expansion related proteins

| | |
|---|---|
| SPRC_HUMAN | SPARC (secreted protein acidic and rich in cysteine, |
| | also known as osteonectin or BM-40) |
| FSTL1_HUMAN | Follistatin-related protein 1 |
| TIMP1_HUMAN | Metalloproteinase inhibitor 1 |
| CSF1R_HUMAN | Macrophage colony-stimulating factor 1 receptor |
| POSTN_HUMAN | Periostin |
| LEG1_HUMAN | Galectin-1 |
| CD166_HUMAN | CD166 antigen |
| FUBP1_HUMAN | Far upstream element-binding protein 1 |

HSC homing related proteins

| | |
|---|---|
| CO3_HUMAN | Complement C3 |
| CO4A_HUMAN | Complement C4-A |
| C1S_HUMAN | Complement C1s subcomponent |
| C1R_HUMAN | Complement C1r subcomponent |

Immune modulation related proteins

| | |
|---|---|
| PGRP2_HUMAN | N-acetylmuramoyl-L-alanine amidase |
| PTX3_HUMAN | Pentraxin-related protein PTX3 |
| VTDB_HUMAN | Vitamin D-binding protein |
| C1RL_HUMAN | Complement C1r subcomponent-like protein |
| LG3BP_HUMAN | Galectin-3-binding protein |
| CD14_HUMAN | Monocyte differentiation antigen CD14 |
| SPON2_HUMAN | Spondin-2 |
| ICAM2_HUMAN | Intercellular adhesion molecule 2 |
| CLUS_HUMAN | Clusterin |
| ICAM1_HUMAN | Intercellular adhesion molecule 1 |
| LYAM1_HUMAN | L-selectin |
| AOC3_HUMAN | Membrane primary amine oxidase |
| DEF1_HUMAN | Neutrophil defensin 1 |

Neuron development related proteins

| | |
|---|---|
| ATRN_HUMAN | Attractin |
| VIME_HUMAN | Vimentin |
| GDN_HUMAN | Glia-derived nexin |
| SAP_HUMAN | Prosaposin |
| SPON2_HUMAN | Spondin-2 |
| PTGDS_HUMAN | Prostaglandin-H2 D-isomerase |
| GFAP_HUMAN | Glial fibrillary acidic protein |
| CADH1_HUMAN | Cadherin-1 |
| CADH2_HUMAN | Cadherin-2 |
| CAD13_HUMAN | Cadherin-13 |
| G6PI_HUMAN | Glucose-6-phosphate isomerase |
| GPC1_HUMAN | Glypican-1 |
| TICN1 HUMAN | Testican-1 |
| PEDF_HUMAN | Pigment epithelium-derived factor |
| CADM1_HUMAN | Cell adhesion molecule 1 |

Metabolic process related proteins

| | |
|---|---|
| BTD_HUMAN | Biotinidase |
| CNDP1_HUMAN | Beta-Ala-His dipeptidase |
| MMP2_HUMAN | 72 kDa type IV collagenase |
| LCAT_HUMAN | Phosphatidylcholine-sterol acyltransferase |
| CBPA4_HUMAN | Carboxypeptidase A4 |
| CPN2_HUMAN | Carboxypeptidase N subunit 2 |
| G3P_HUMAN | Glyceraldehyde-3-phosphate dehydrogenase |
| CATD_HUMAN | Cathepsin D |
| VNN1_HUMAN | Pantetheinase |
| ENOA_HUMAN | Alpha-enolase |
| BST1_HUMAN | ADP-ribosyl cyclase/cyclic ADP-ribose hydrolase 2 |
| DIAC_HUMAN | Di-N-acetylchitobiase |
| LAMP2_HUMAN | Lysosome-associated membrane glycoprotein 2 |
| PHLD_HUMAN | Phosphatidylinositol-glycan-specific phospholipase D |
| PEPD_HUMAN | Xaa-Pro dipeptidase |
| PGBM_HUMAN | Basement membrane-specific heparan sulfate |
| | proteoglycan core protein |
| AMPN_HUMAN | Aminopeptidase N |
| NPC2_HUMAN | Epididyrnal secretory protein E1 |
| THIO_HUMAN | Thioredoxin |
| CBPB2_HUMAN | Carboxypeptidase B2 |
| CBPN_HUMAN | Carboxypeptidase N catalytic chain |
| TPIS_HUMAN | Triosephosphate isomerase |
| MANBA_HUMAN | Beta-mannosidase |
| GNS_HUMAN | N-acetylglucosamine-6-sulfatase |
| GGH_HUMAN | Gamma-glutamyl hydrolase |
| FAAA_HUMAN | Fumarylacetoacetase |
| 5NT3B_HUMAN | 7-methylguanosine phosphate-specific 5~- |
| | nucleotidase |
| ANXA2_HUMAN | Annexin A2 |

Cellular component related proteins

| | |
|---|---|
| CO6A1_HUMAN | Collagen alpha-1(VI) chain |
| CO1A2_HUMAN | Collagen alpha-2(I) chain |
| CO1A1_HUMAN | Collagen alpha-1(I) chain |
| CO3A1_HUMAN | Collagen alpha-1(III) chain |
| COSA1_HUMAN | Collagen alpha-1(V) chain |
| CO4A1_HUMAN | Collagen alpha-1(IV) chain |
| PGS1_HUMAN | Biglycan |
| ACTB_HUMAN | Actin, cytoplasmic 1 |
| LUM_HUMAN | Lumican |
| FINC_HUMAN | Fibronectin |
| BGH3_HUMAN | Transforming growth factor-beta-induced protein |
| | ig-h3 |
| ACTN1_HUMAN | Alpha-actinin-1 |
| PGS2_HUMAN | Decorin |
| PROF1_HUMAN | Profilin-1 |
| FBN1_HUMAN | Fibrillin-1 |
| TAGL_HUMAN | Transgelin |
| ECM1_HUMAN | Extracellular matrix protein 1 |
| TPM1_HUMAN | Tropomyosin alpha-1 chain |
| VINC_HUMAN | Vinculin |

Vesicle transport proteins

| | |
|---|---|
| 1433Z_HUMAN | 14-3-3 protein zeta/delta |
| CSTN1_HUMAN | Calsyntenin-1 |
| 1433E_HUMAN | 14-3-3 protein epsilon |
| NPC2_HUMAN | Epididymal secretory protein E1 |

In some embodiments, the umbilical cord mesenchymal stem cell conditioned medium comprises at least one HSC expansion related proteins selected from the following group: secreted protein acidic and rich in cysteine (SPARC), Follistatin-related protein 1, Metalloproteinase inhibitor 1, Macrophage colony-stimulating factor 1 receptor, Periostin, Galectin-1, CD166 antigen, Far upstream element-binding protein 1, or any combination thereof.

### Supplemental Components

In the embodiments of the disclosure, various supplemental components may be used in the serum-free culture medium, including vitamin C, vitamin E, estradiol (E2), and transferrin (TF). In a previous study, vitamin C and vitamin E have been provided as medical nutrition therapy to adult hematopoietic stem cell transplantation patients to minimize conditioning regimen-inducing toxicities. Nutr. Clin. Pract., October 2012, 27: 655-660.

As used herein, the term “vitamin C (Vit. C)” refers to L-ascorbic acid, either synthetic or natural, the bio-available form, or a derivative thereof. In an embodiment, the concentration of Vit. C is in a range of 50-375 μM, preferably 100-300 μM, more preferably 200-300 μM. In a preferred embodiment, the concentration of Vit. C is 250 μM.

As used herein, the term “vitamin E (Vit. E)” refers to all tocopherols (i.e. α-, β- and γ-tocopherol in all steric forms), either synthetic or natural, the bio-available form, or a derivative thereof. In an embodiment, α-tocopherol is preferred for the purpose of the present invention. In an embodiment, the concentration of Vit. E is in a range of 2-20 μM, preferably 2-15 μM, more preferably 2-10 μM. In a preferred embodiment, the concentration of Vit. E is 2 μM.

In an embodiment, the concentration of estradiol (E2) is in a range of 10⁻⁹-10⁻⁸ M. In a preferred embodiment, the concentration of estradiol is 10⁻⁹ M.

In an embodiment, the concentration of transferrin (TF) is in a range of 10-100 μg/ml, preferably 10-80 μg/ml, more preferably 10-50 μg/ml. In a preferred embodiment, the concentration of transferrin is 30 μg/ml.

### EXAMPLES

The following experimental examples were performed to evaluate the various different factors that may affect the expansion of hematopoietic stem cells.

### Example 1

### Assessing HSC Culture Media

In order to assess various media for ex vivo expansion of HSC, IMDM with the necessary cytokines, 5% cord serum, and a feeder layer were used as a control. Two media, SCGM and X-VIVO 15, were tested in the absence of serum (but in the presence of the same cytokines and feeder layers) to see whether they can be used as serum-free media. The experimental procedures are as follows.

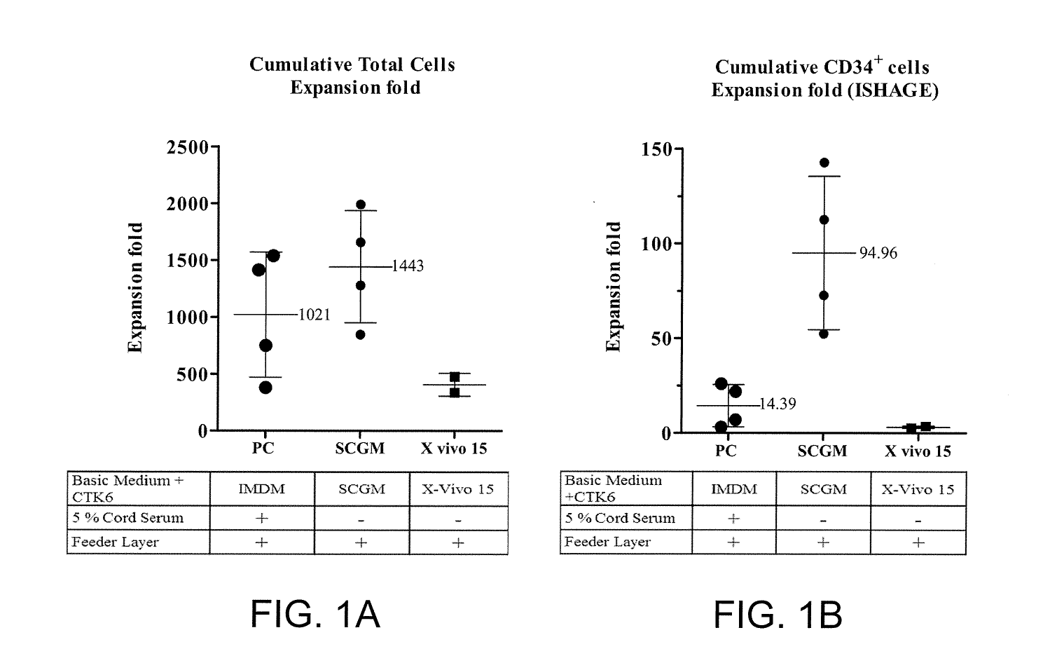

UC-MSC were seeded and cultured in complete culture medium (containing 10% human cord serum and DMEM) as feeder cells in a T-12.5 flask at day −1. At day 0, CD34+ HSC are thawed and co-cultured with UC-MSC feeder cells for 12 days at a cell density of 2.5×10⁴ cells/mL, using different culture medium as follows: (1) positive control (PC) group: IMDM containing 5% cord serum, 6 cytokines and hydrocortisone (10⁻⁶ M); (2) SCGM group: serum-free SCGM containing 6 cytokines and hydrocortisone (10⁻⁶ M); and (3) X-VIVO 15 group: serum-free X-VIVO 15 containing 6 cytokines and hydrocortisone (10⁻⁶ M). During the 12-day culture, 50% culture medium and newly prepared UC-MSC feeder are replenished every 4 days, and the cells (including CD34+ cells) are maintained at the cell density of 2.5-5×10⁴ cells/ml. The six (6) cytokines used above include recombinant human stem cell factor (rh SCF, 20 ng/ml), recombinant human thrombopeietin (rh TPO, 20 ng/ml), recombinant human hematopoietic growth factor Flt3 ligand (rh Flt3L, 50 ng/ml), recombinant human interleukin 3 (rh IL-3, 10 ng/ml), recombinant human interleukin 6 (rh IL-6, 10 ng/ml), and recombinant human granulocyte colony stimulating factor (rh G-CSF, 1 ng/ml). At day 12, cumulative total cell expansion folds and CD34+ (ISHAGE) expansion folds are calculated, and the results are shown in FIG. 1A and FIG. 1B. CD34+ cells were quantified in accordance with the International Society of Hematotherapy and Graft Engineering (ISHAGE) guidelines (Sutherland et al., J. Hematother., 1996, June: 5(3): 213-26).

As shown in FIG. 1A and FIG. 1B, among the tested media, SCGM, in the absence of serum, could support HSC expansion much better than the control IMDM, while X-VIVO 15 is less effective. Furthermore, in the presence of a feeder layer, SCGM is a good serum-free medium not only for the expansion of total cells, but even more so for the expansion of CD34+ cells. It is known that most proliferative HSCs are CD34+ cells. Therefore, the ability to sustain the expansion of CD34+ cells is more important than sustaining total cell growth. For this reason, SCGM was chosen as the base medium for the serum-free culture media of the invention.

As noted above, ex vivo growth of HSC requires various cytokines and factors contributed by other cells or tissues. One hypothesis is that true HSC are in essence fixed tissue cells. They exist together with other supporting tissues/cells, and the microenvironments provided by these supporting tissues/cells enable HSC to self-renew, without differentiation and maturation. In this regard, stromal cells are shown to provide a wide range of environmental signals, mediated by cytokines, extracellular matrix proteins and adhesion molecules, that can control proliferation, survival and differentiation of hematopoietic progenitor and stem cells. Thus, a feeder layer, which contain stromal cells, in the ex vivo growth of HSC may provide any of these factors.

However, the use of a tissue or cells as a feeder layer is undesirable because it may introduce contamination or cause adverse immune responses. Inventors of the present invention have found that conditioned media from umbilical cord mesenchymal stem cells (UC-MSC) can replace the feeder layer in supporting ex vivo expansion of HSC. The experiments supporting these founding will be explained in detail in the latter examples.

### Example 2

### Assessing the Effects of Supplements

The effects of adding four supplements to the growth media containing feeder layers were evaluated. The experimental procedures are as follows.

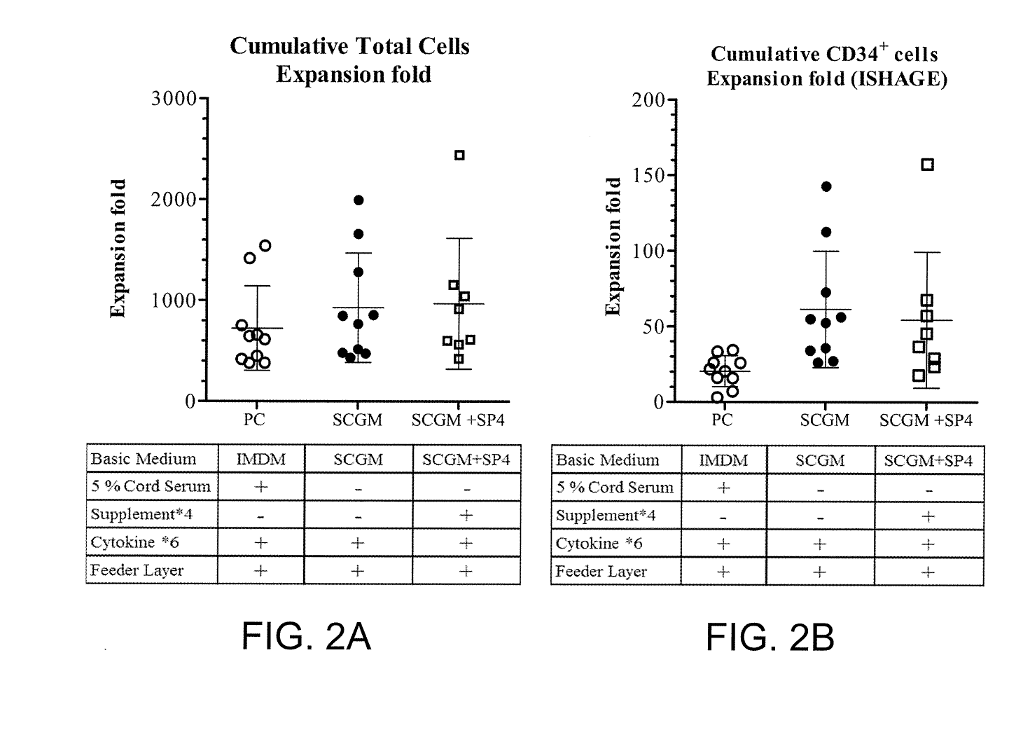

UC-MSC were seeded and cultured in complete culture medium (containing 10% human cord serum and DMEM) as feeder cells in a T-12.5 flask at day −1. At day 0, CD34+ HSC are thawed and co-cultured with the UC-MSC feeder for 12 days with a cell density of 2.5×⁴ cells/mL, using different culture medium as follows: (1) positive control (PC) group: IMDM containing 5% cord serum, 6 cytokines and hydrocortisone; (2) SCGM group: SCGM containing 6 cytokines and hydrocortisone; and (3) SCGM+SP4 group: SCGM containing 6 cytokines, hydrocortisone and 4 supplements. The 6 cytokines and hydrocortisone used herein are the same as described in example 1. The 4 supplements (also referred as SP4 or supplements*4) include vitamin C (250 μ), vitamin E (2 μestradiol (10⁻⁹ M), and transferrin (30 ug/ml). During the 12-day culture, 50% culture medium and newly prepared UC-MSC feeder layer were replenished every 4 days, and the cells including CD34+ cells are maintained at a cell density of 2.5-5×⁴ cells/mL. At day 12, cumulative total cell expansion folds and CD34+ (ISHAGE) expansion folds are calculated. The results are shown in FIG. 2 A and FIG. 2B.

As shown in FIG. 2A and FIG. 2B, in the presence of a feeder layer, the four supplements do not have any significant effects on total cell expansion and CD34+ cell expansion. It is possible that in the presence of a feeder layer, certain factors secreted by the feeder layer have similar effects as the four supplements. Therefore, addition of the four supplements on top of these factors do not reveal any further enhancement.

### Example 3

### Replacing Feeder Layer with SF-UCM

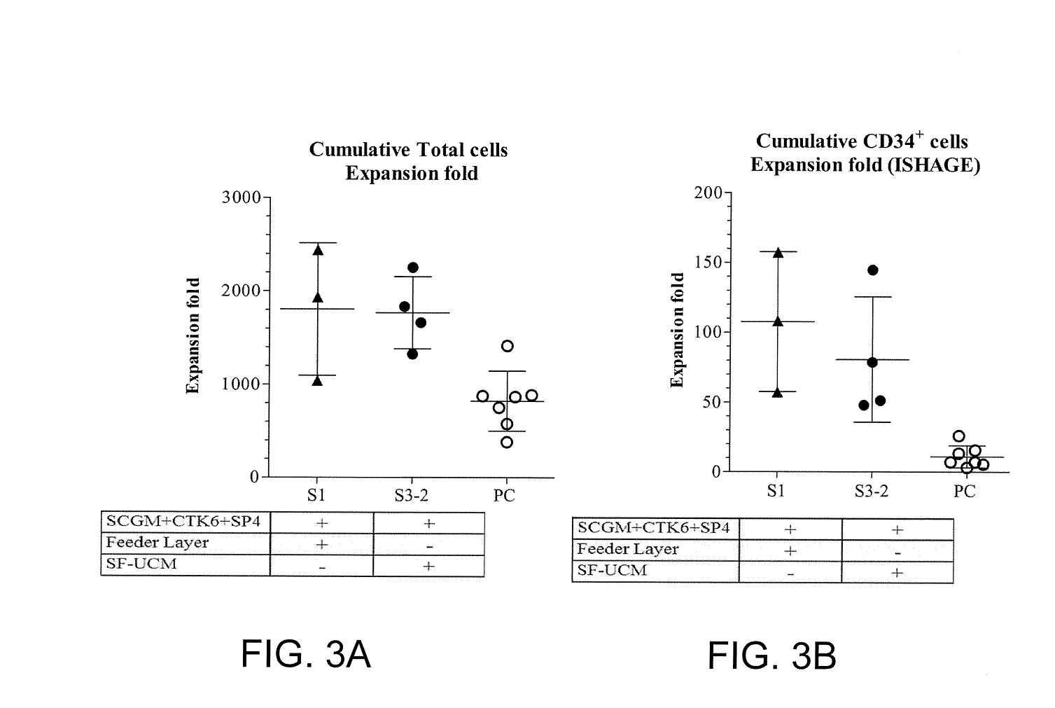

To test potential replacement for the feeder layers, we have tested a serum-free umbilical cord mesenchymal stem cell conditioned medium (SF-UCM) from UC-MSC. The serum-free umbilical cord mesenchymal stem cell conditioned medium is for example produced by the method described above, comprising the steps of: (a) culturing an umbilical cord mesenchymal stem cell in a serum-free cell culture medium (e.g. serum-free SCGM), and (b) isolating the conditioned cell culture medium. The results of replacing the feeder layers with SF-UCM are shown in FIG. 3A and FIG. 3B.

In FIG. 3A and FIG. 3B, the positive control (PC) group is in IMDM as described above. The PC & S1 groups shown in FIG. 3A and 3B are grown as follows: At day 0, CD34+ HSC are thawed and co-cultured with UC-MSC feeder for 12 days with a cell density of 2.5×10⁴ cells/ml, using 5% CS (cord serum)/IMDM medium containing the 6 cytokines and hydrocortisone for PC group or using SCGM containing 6 cytokines, hydrocortisone and 4 supplements for S1 group.

The S3-2 group was grown as follows: At day 0, CD34+ HSC are thawed and cultured in a culture medium mixture of 50% (v/v) SF-UCM and 50% (v/v) fresh SCGM containing 6 cytokines, hydrocortisone and 4 supplements with a cell density of 2.5×10⁴ cells/ml for 12 days. The 6 cytokines, hydrocortisone and 4 supplements used herein are the same as described in example 2.

During 12-day culture, 50% culture medium is replenished as well as newly prepared UC-MSC feeder every 4 days, and CD34+ cells are maintained at a cell density of 2.5-5×10⁴ cells/ml. At day 12, cumulative total cell expansion folds and CD34+ expansion folds are calculated.

From the results shown in FIG. 3, the SF-UCM can replace the feeder layer without discernable impact on the expansion of total cells. In addition, the SF-UCM can also replace the feeder layer in CD34+ cell expansion, albeit with a slightly lower effectiveness. The above results indicate that SF-UCM is a good substitute for a feeder layer.

### Example 4

### Comparison of SF-UCM and Concentrated SF-UCM as a Substitute for a Feeder Layer

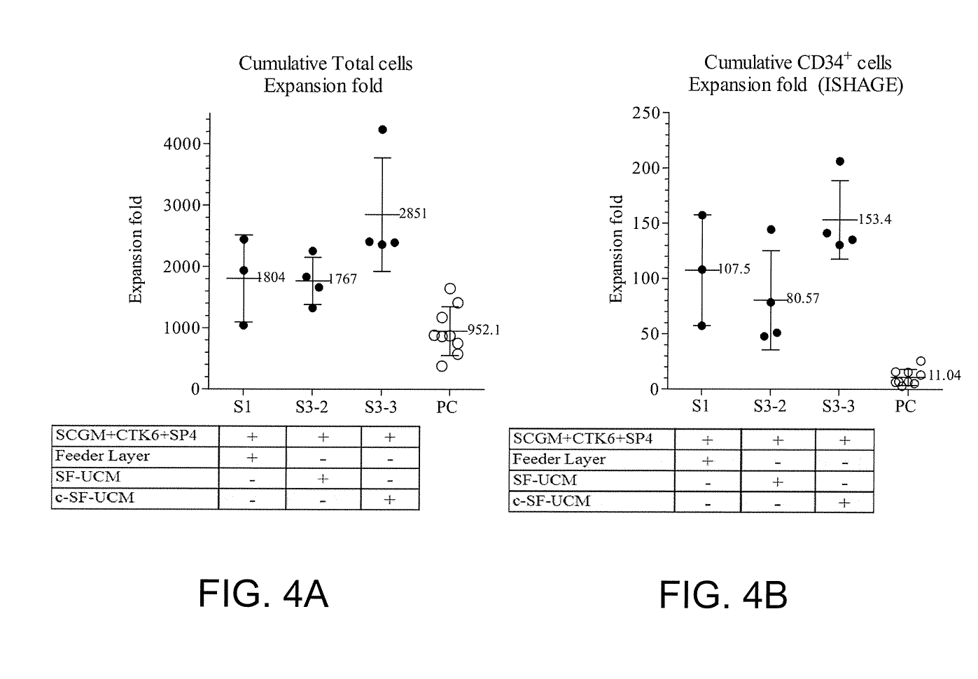

The following experiment is performed to evaluate the effect of replacing feeder layers with SF-UCM or concentrated SF-UCM. The SF-UCM obtained in Example 3 was further concentrated with a 5-10 kDa cut-off membrane (preferably a 5 kDa cut-off membrane) to obtain 10 times concentrated (in volume) conditioned medium. The concentrated conditioned medium was filtered with a 0.22 μm filter, and the filtrate is collected to obtain the desired con. SF-UCM (abbreviated as “c-SF-UCM”). The results of replacing the feeder layers with SF-UCM or con. SF-UCM are shown in FIG. 4A and FIG. 4B.

In FIG. 4A and 4B, the PC, Sl, and S3-2 groups are the same as described in Example 3. The S3-3 group is as follows: At day 0, CD34+ HSC are thawed and cultured in a culture medium mixture of 5% (v/v) concentrated SF-UCM and 95% (v/v) SCGM containing 6 cytokines, 4 supplements, and hydrocortisone with a cell density of 2.5×10⁴ cells/ml for 12 days. The 6 cytokines, hydrocortisone and 4 supplements used herein are the same as described in example 2. The above medium mixture is 50% replenished every 4 days and the cells (including CD34+ cells) are maintained at a cell density of 2.5-10×10⁴ cells/ml. At day 12, cumulative total cell expansion folds and CD34+ (ISHAGE) expansion folds are calculated.

As shown in the results presented in FIG. 4A and FIG. 4B, concentrated SF-UCM is more effective than SF-UCM in supporting the total cell expansion, as well as CD34+ cell expansion. In fact, the concentrated SF-UCM is even more effective than the feeder layer. That is, con. SF-UCM revealed the greatest stern cell expansion as compared with SF-UCM or when feeder layer is used. The fact that concentrated SF-UCM is better than the feeder layer is unexpected. These results may suggest that certain factors in the conditioned medium at higher concentrations (as compared to the concentrations produced by a feeder layer) have better activities.

### Example 5

### Combination of Four Supplements and Concentrated SF-UCM Have a Great Improvement Effect on HSC Expansion

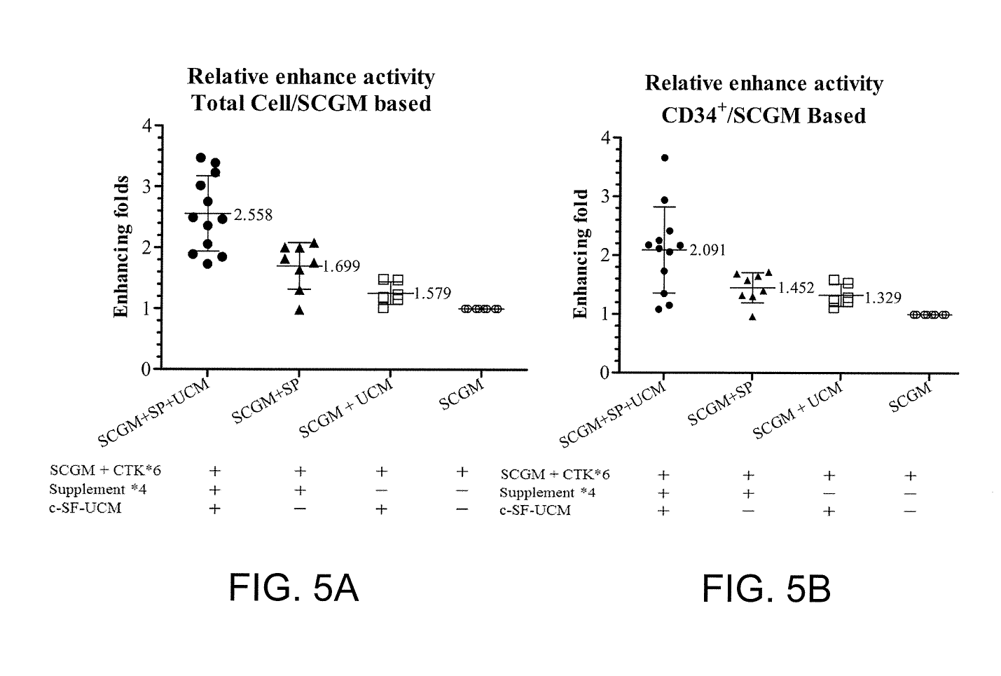

As noted above, four supplements (vitamin C, vitamin E, estradiol, and transferrin) do not produce measurable enhancements in the expansion tests when using a feeder layer simultaneously. Since the concentrated SF-UCM has a superior activity as shown in Example 4, we further tested the effects of the four supplements using the concentrated SF-UCM (abbreviated as “c-SF-UCM”). The results of these tests are shown in FIG. 5A and FIG. 5B.

At day 0, CD34+ HSC are thawed and cultured in a corresponding culture medium in the following groups: (1) SCGM+SP+UCM group: a culture medium mixture of 5% (v/v) c-SF-UCM and 95% (v/v) SCGM containing 6 cytokines, 4 supplements and hydrocortisone; (2) SCGM+SP group: SCGM containing 6 cytokines, 4 supplements and hydrocortisone; (3) SCGM+UCM group: a culture medium mixture of 5% (v/v) c-SF-UCM and 95% (v/v) SCGM containing 6 cytokines and hydrocortisone; and (4) SCGM group: SCGM containing 6 cytokines and hydrocortisone. The 6 cytokines, hydrocortisone and 4 supplements used herein are the same as described in example 2.

The cells are cultured at a cell density of 2.5×10⁴ cells/ml. The above medium mixture is 50-80% replenished every 4 days and the cells (including CD34+ cells) are maintained at a cell density of 2.5-10×10⁴ cells/ml. At day 12, cumulative total cell expansion folds and CD34+ (ISHAGE) expansion folds are calculated.

FIG. 5A and FIG. 5B shows the cumulative total cell expansion folds and CD34+ (ISHAGE) expansion folds, as compared with the SCGM group. As shown in FIG. 5A and FIG. 5B, in the presence of SCGM and 6 cytokines (but in the absence of a feeder layer), the four supplements and c-SF-UCM individually can enhance the expansions of both total cells and CD34+ cells.

The fact that the four supplements can enhance the cell expansion is unexpected and is in contrast to the results seen in the presence of a feeder layer (see FIG. 2A and FIG. 2B). As mentioned above, it is possible that certain factors secreted by the feeder layer have similar effects as the four supplements. Therefore, the effect of the four supplements in enhancing cell expansion was not observable when feeder layers are used, and is apparent when the feeder layer is replaced with c-SF-UCM. That is, when the feeder layer is absent, the four supplements can substitute for the missing factors, thereby producing enhancements.

When both the four supplements and c-SF-UCM are added in the same culture, the expansion fold of cumulative total cell or CD34+ cell is significantly increased. It is likely that the four supplements and the factors in the c-SF-UCM contribute to different stages in the cell expansion pathways, thereby their combination have a great improvement effect on HSC expansion. Thus, the four supplements and the c-SF-UCM work in a synergistic manner.

### Example 6

### HSC Expansion Does Not Impact Colony Forming Units (CFU)

Ex vivo expansion of stem cells requires symmetric divisions, wherein both daughter cells retain properties of stem cells. One problem encountered in ex vivo expansion of HSC is the possible differentiation and maturation of the expanded cells. The differentiated cells may not develop into the desired types of cells after transplantation.

To detect the committed hematopoietic progenitors, uncultured CD34+ cells of day 0 and cultured CD34+ cells of day 12 were seeded in cytokine-supplemented MethoCult methylcellulose medium (Stemcell Technology, Vancouver, Canada) in 35 mm dishes at a concentration of 100 and 5000 cell/ml, respectively. After 14 days of incubation at 37 ° C. in a moisture-saturated atmosphere, 20% O₂ and 5% CO₂, the total colony forming units (CFUs) including CFU-G, CFU-M, CFU-GM, CFU-E and BFU-E were counted using an inverted microscope. Cumulative CFU expansion folds of each group were normalized to day 0 uncultured CFU total numbers and shown as relative expansion folds.

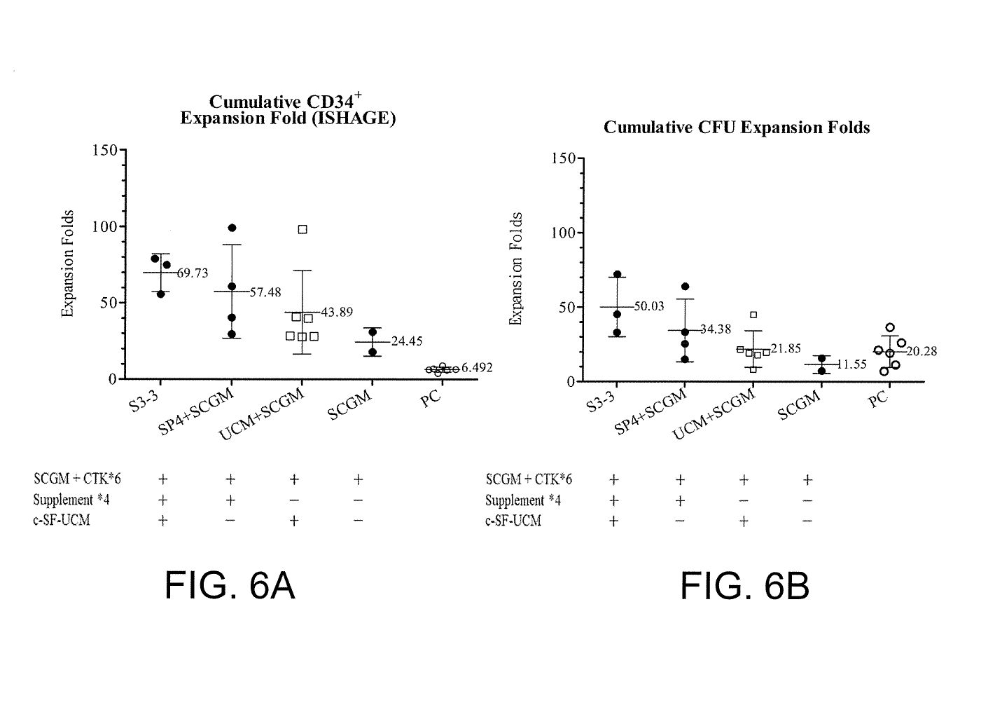

As shown in FIG. 6A and 6B, the cumulative CFU expansion folds parallel the cumulative CD34+ cell expansion folds (ISHAGE), indicating that the expanded CD34+ cells maintained the stem cell properties.

### Example 7

### Testing Effects of Individual Components in the Four Supplement Mixture

As noted in the example above, the four supplements enhanced cell expansion in the media of the invention, in the absence of a feeder layer. To further understand the roles of the supplements, we have examined the roles of each supplement.

In this experiment, the PC group and S3-3 group is the same as the previous examples. The culture medium is 50% replenished every 4 days, and cells are maintained at a cell density of 2.5-5×10⁴ cells/ml.

Other groups are prepared as follows: At day 0, CD34+ HSC are thawed and cultured in a corresponding culture medium in the following groups: (1) SP3 in UCM group: a culture medium mixture of 5% (v/v) c-SF-UCM and 95% (v/v) SCGM containing 6 cytokines, 3 supplements (estradiol E2+Vit. C+Vit. E) and hydrocortisone; (2) SP3 in SCGM group: SCGM containing 6 cytokines, 3 supplements (estradiol E2+Vit. C+Vit. E) and hydrocortisone; (3) UCM group: a culture medium mixture of 5% (v/v) c-SF-UCM and 95% (v/v) SCGM containing 6 cytokines and hydrocortisone; and (4) SCGM group: SCGM containing 6 cytokines and hydrocortisone. The 6 cytokines, hydrocortisone and supplements used herein are the same as described in example 2. The cells are cultured at a cell density of 2.5×10⁴ cells/ml. The above medium mixture is 80% replenished every 4 days, and the cells (including CD34+ cells) are maintained at a cell density of 2.5-10×10⁴ cells/ml. At day 12, cumulative total cell expansion folds and CD34+ (ISHAGE) expansion folds are calculated.

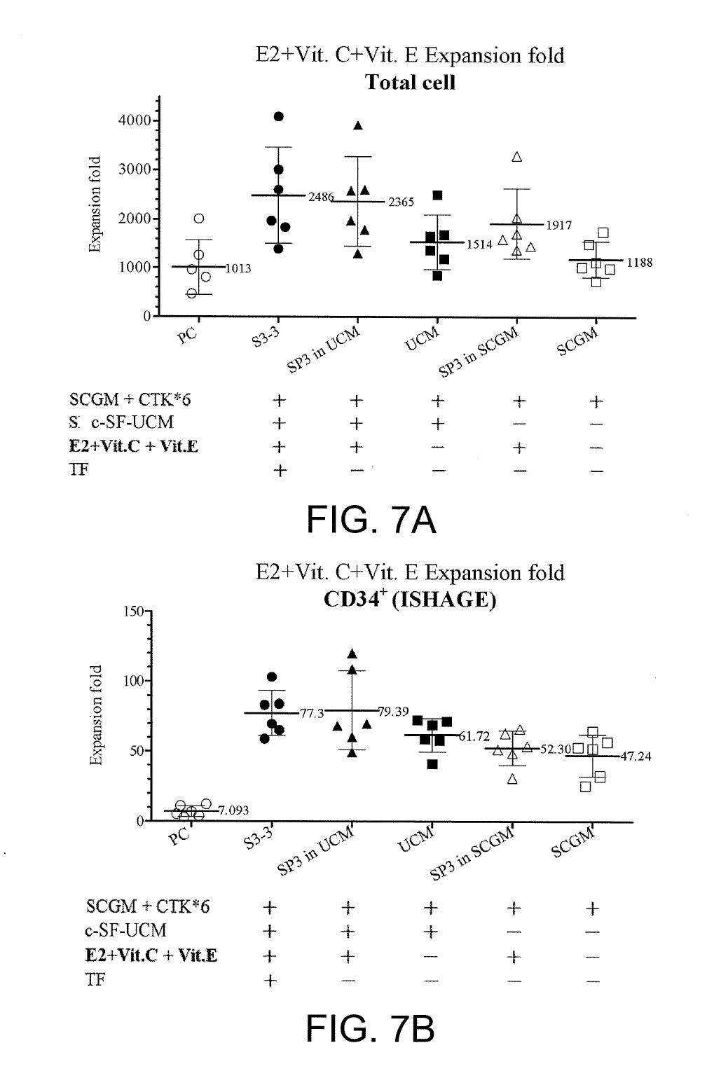

FIG. 7A and FIG. 7B shows the results of hematopoietic stem cell expansion by evaluating the effect of individual supplements. The roles of each supplement were examined and the results indicates that transferrin (TF) can be left out. As shown in FIG. 7A and FIG. 7B, the remaining three (3) supplements, vitamin C, vitamin E, and estradiol (E2), when added can enhance cell expansion folds. Further addition of transferrin (TF) does not further increase the enhancement to any appreciable extent.

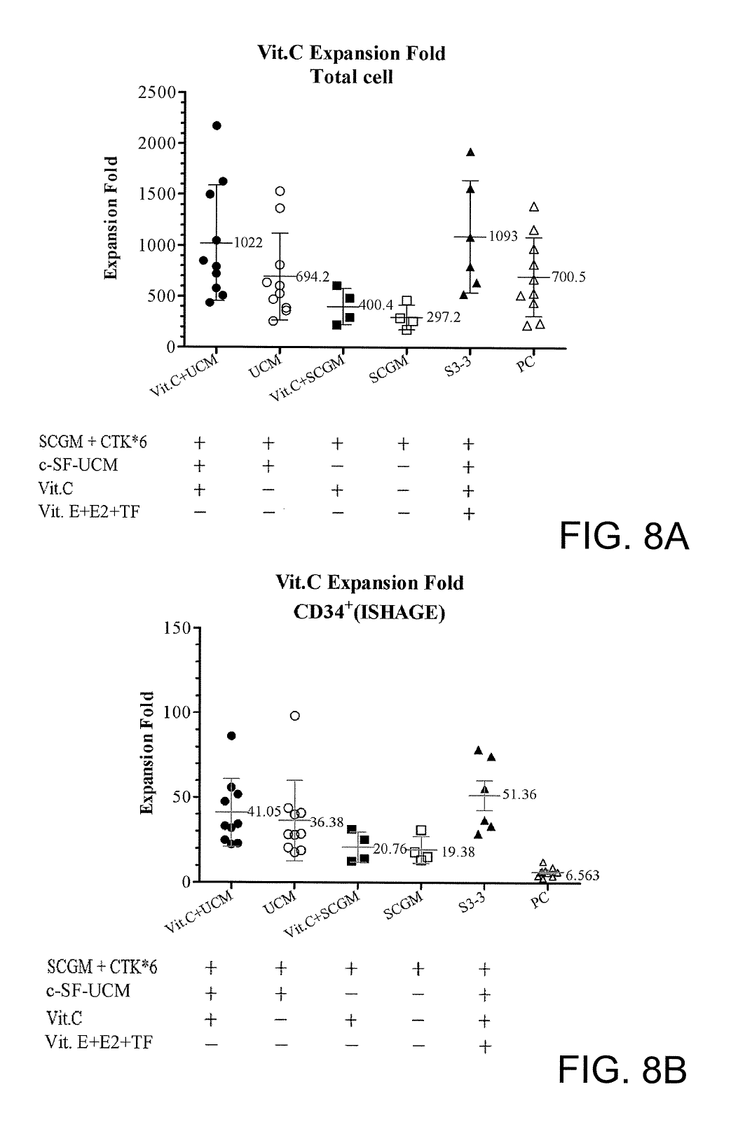

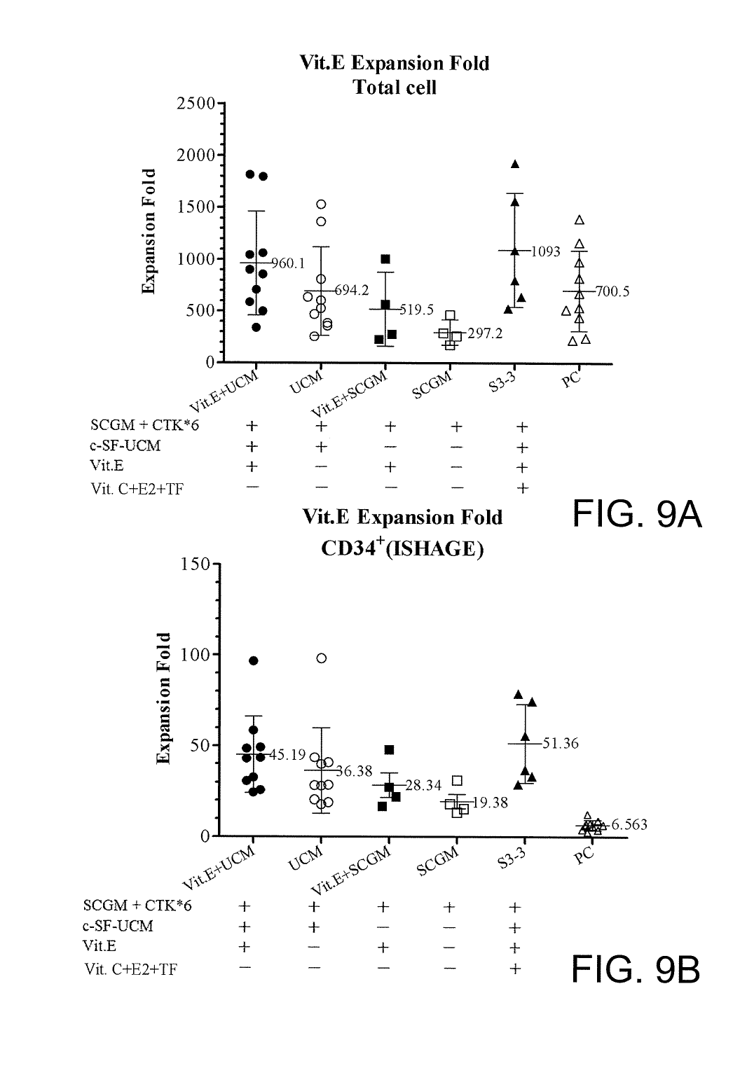

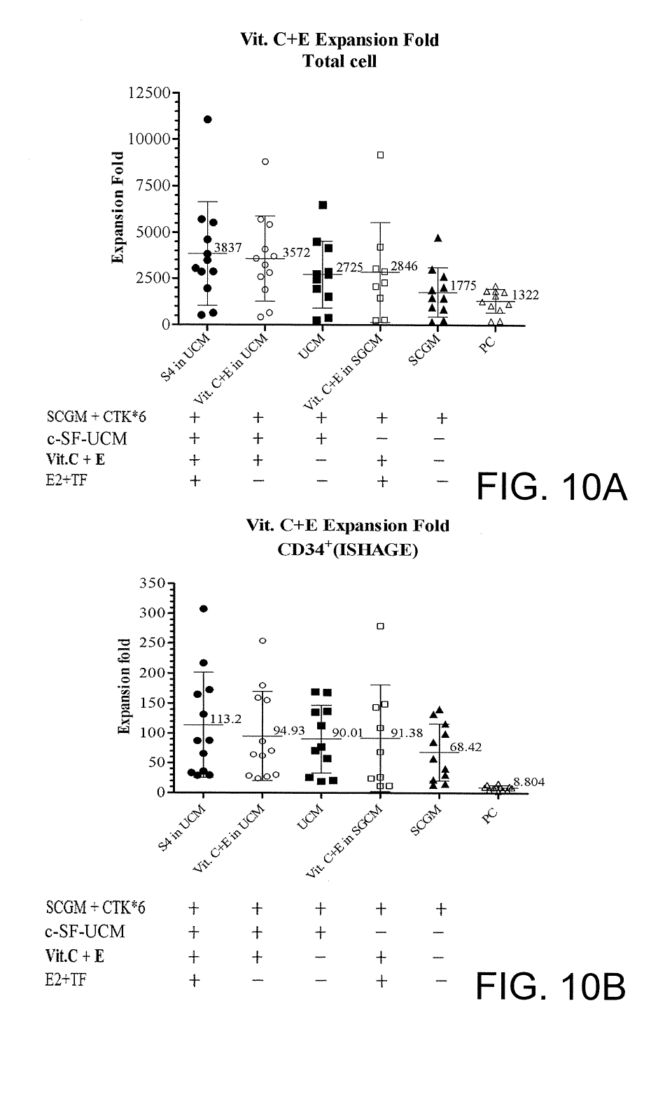

FIG. 8A and FIG. 8B shows the results of hematopoietic stem cell expansion by evaluating the effect of vitamin C. The results indicate that vitamin C alone can support the expansion of both the total cells and the CD34+ cells. FIG. 9A and FIG. 9B shows the results of hematopoietic stem cell expansion by evaluating the effect of vitamin E. The results indicate that vitamin E alone is also sufficient in supporting the expansion of both the total cells and the CD34+ cells. FIG. 10A and FIG. 10B shows the results of hematopoietic stem cell expansion by evaluating the effect of combining vitamin C and vitamin E. The results indicate that the combination of both vitamin C and vitamin E also produce good results in supporting the expansion of cells.

### Example 8

### Different Amounts of Medium Replenishments

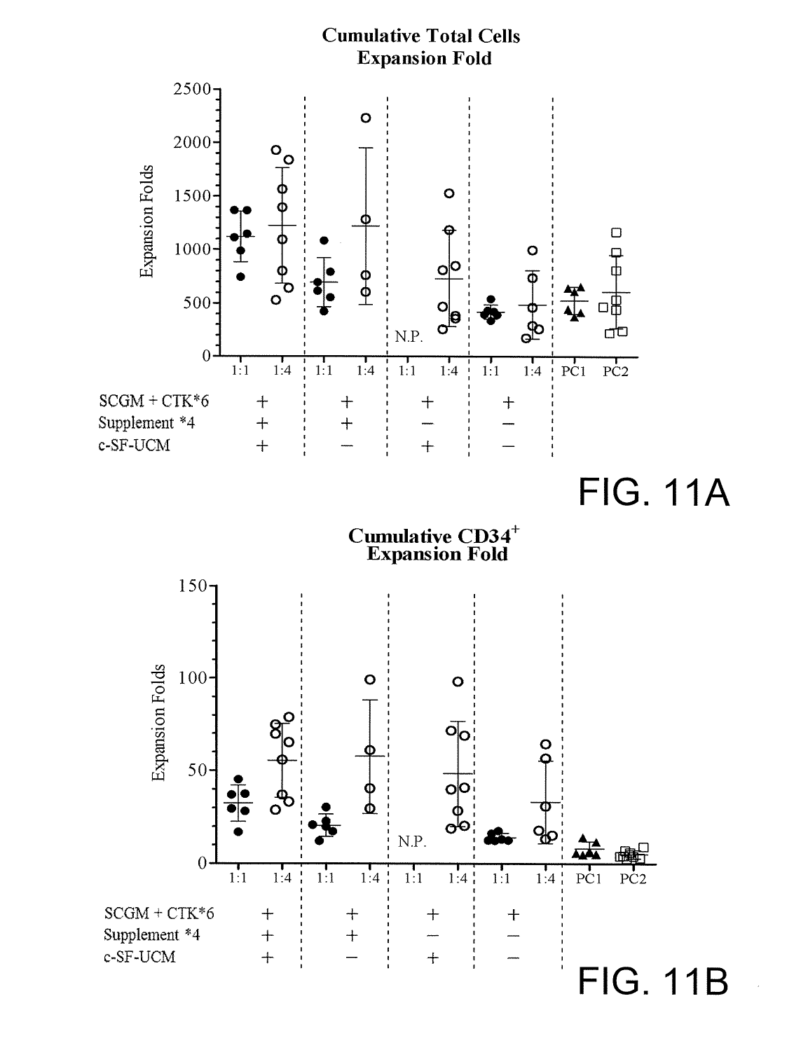

The above results indicate that the serum and the feeder layer in the culture media can be replaced with judicially selected factors and supplements for ex vivo HSC expansion. To further investigate the culture protocols, the culture procedures were varied. The experimental conditions are the same as described for the above experiments, except that the amounts of media replenished every 4 days were changed. One group was replenished 50% (1:1) and the other group was replenished 80% (1:4) every 4 days. The results are shown in FIG. 11A and FIG. 11B.

From the results shown in FIG. 11A and FIG. 11B, when different amounts of the media were replenished, 80% replenishment every 4 days produced better results than 50% replenishments every 4 days. It is clear that the cells would benefit from more fresh media.

### Example 9

### Cytokines Required for HSC Expansion

In the above experiments, six (6) cytokines are included in the medium: rh SCF, rh TPO, rh Flt3L, rh IL-3, rh IL-6, and rh G-CSF. To test whether all these cytokines are needed, the following experiments were performed to leave out some of the cytokines.

In the example, UC-MSC were seeded and cultured with complete culture medium (containing 10% human cord serum and DMEM) as feeder cells in a T-12.5 flask at day -1. At day 0, CD34+ HSC are thawed and co-cultured with UC-MSC feeder for 6 days in T-12.5 flasks with a cell density of 2.5×10⁴ cells/mL, using 2 ml of 5% CS/IMDM containing 3, 5 or 6 cytokines, and hydrocortisone. The 3 cytokines group (Cytokine*3) include rh SCF, rh TPO, and rhF1t3L. The 5 cytokines group (Cytokine*5) include rh SCF, rh TPO, rhFlt3L, IL-3, and IL-6. The 6 cytokines group (Cytokine*6) include rh SCF, rh TPO, rh Flt3L, rh IL-3, rh IL-6, and rh G-CSF. During cell cultures, additional 3 ml culture medium mixture are added. At day 6, cumulative total cell expansion folds and CD34+ (ISHAGE) expansion folds are calculated.

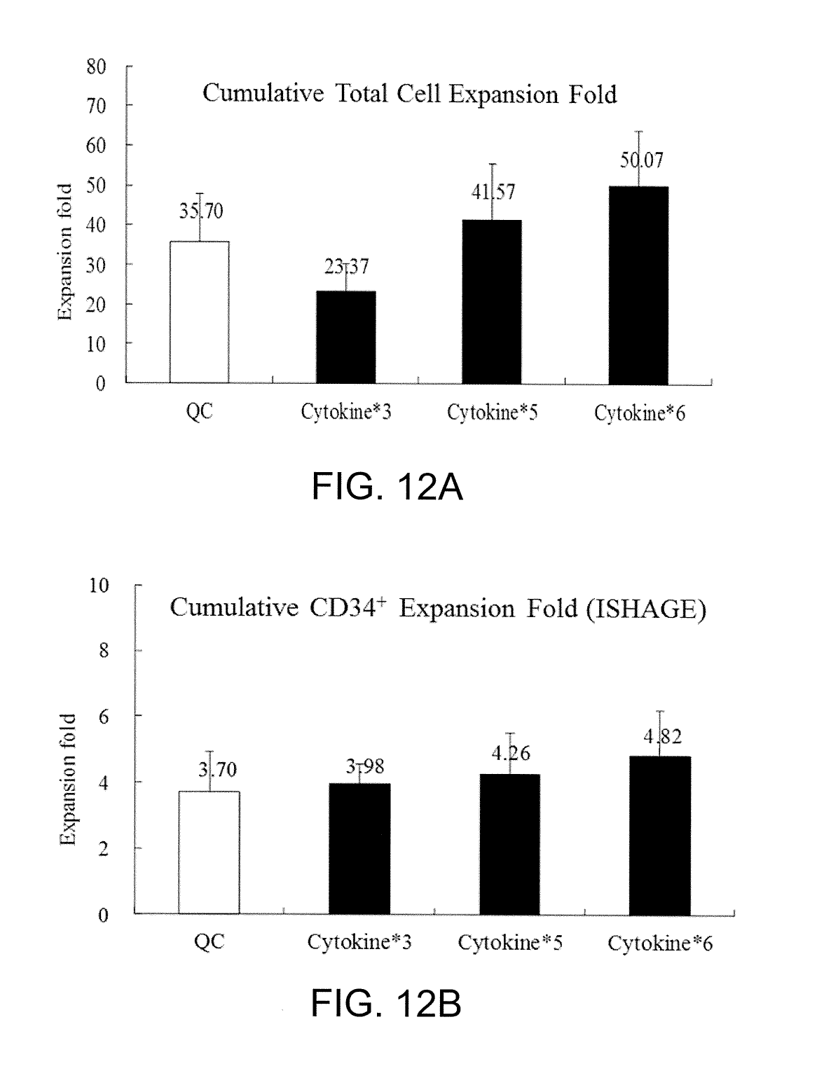

The QC group: HSC (including CD34+ cells) are co-cultured with COH275 feeder cells using COH medium (15% FBS/Myelocult H5100+IMDM) with 3 cytokines including rhSCF, rhTPO, and rhFlt3L. The cell density is 2.5×10⁴ cells/mL. At day 3 and day 5, 3 ml culture medium mixture is added. The results from these tests are shown in FIG. 12A and FIG. 12B.

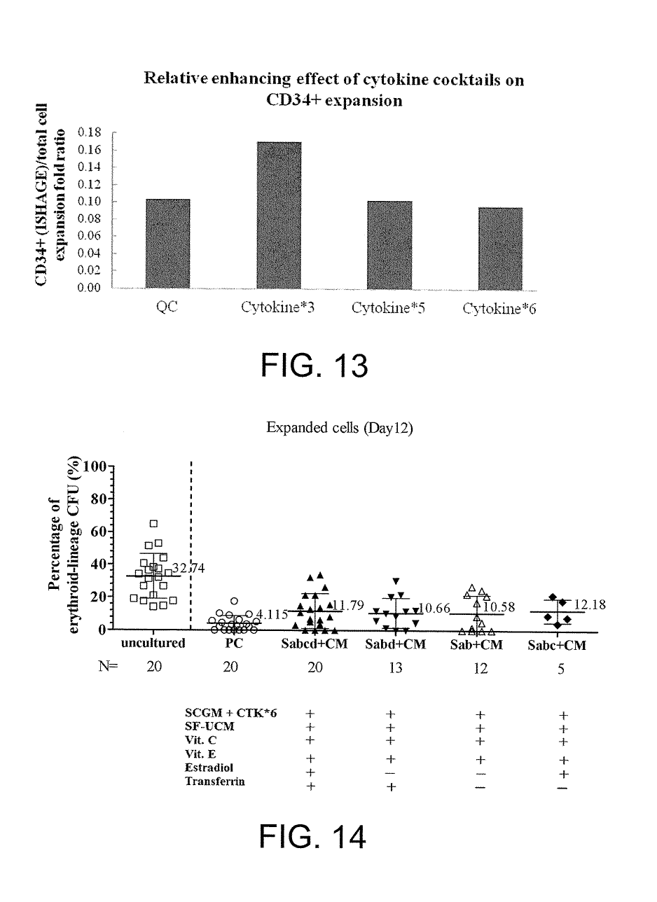

From the results, it is clear that the total cell expansion and CD34+ cell expansion both benefit from more cytokines: 6 cytokines >5 cytokines >3 cytokines. However, the preference for more cytokines is more apparent with the total cell expansion, whereas the preference is less apparent for the CD34+ cells. This observation suggests that the non-CD34+ cells in the total cell population would benefit more with more cytokines. This is readily apparent from FIG. 13, which shows the relative expansion of the CD34+ cells relative to the total cell expansion. In this experiment, it is interesting to note that the 3 cytokines seem to be sufficient to support the CD34+ cell expansion, while the additional cytokines (in the 5 cytokines and 6 cytokines groups) seem to benefit more for the non-CD34+ cells and slightly enhance CD34+ cells expansion than 3 cytokines.

### Example 10

### Erythroid-Lineage Cells in the Expanded Cells

Ex vivo HSC expansion is known to produce some differentiated cells and some committed cells for certain lineages. To detect the committed hematopoietic progenitors, uncultured CD34+ cells of day 0 and cultured CD34+ cells of day 12 were seeded in cytokine-supplemented MethoCult methylcellulose medium (Stemcell Technology, Vancouver, Canada) in 35 mm dishes at a concentration of 100 and 5000 cells/ml, respectively.

After 14 days of incubation at 37 ° C. in a moisture-saturated atmosphere, with 20% O₂ and 5% CO₂, the total CFUs including CFU-G, CFU-M, CFU-GM, CFU-E and BFU-E were counted using an inverted microscope. Percentages of erythroid-lineage CFU are calculated using the following formula: (BFU-E+CFU-E)/(BFU-E+CFU-E+CFU-GM+CFU-G+CFU-M) *100%. The abbreviations are as follows: BFU-E is the burst-foiiiiing unit-erythroid; CFU-E is the colony-forming unit-erythroid; CFU-GM is the colony-forming unit-granulocyte, macrophage; CFU-G is the colony-forming unit-granulocyte; and CFU-M is the colony-forming unit-macrophage.

As shown in FIG. 14, cells expanded in media of the invention produce higher percentages of erythroid-lineage CFU, as compared to the positive control group (PC). These results indicate that media of the invention can support expansion of HSC to produce sufficient percentages of cells that retain the erythroid-lineage progenitor cell properties. However, when compared with the uncultured calls, cells expanded in media of the invention do not produce higher percentages of erythroid-lineage CFU. These results suggest that during expansion, some HSC might have become committed to other cell lineages.

From the Examples above, it is clear that the serum-free culture medium of the invention is capable of supporting ex vivo expansions of HSC. A method of the invention may comprise growing HSC in any of the medium of the invention, which contain a base medium, cytokines, defined supplements, and a condition medium obtained from a serum-free culture of umbilical cord mesenchymal stem cells.

Advantages of embodiments of the invention may include one or more of the following. It is easier to implement quality control for the active components and critical materials due to the absence of serum and other tissues (feeder layers). This invention can avoid potential contaminations or adverse immune responses, improve safety of cell therapy products and is easy to scale-up for GMP cell production.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A serum-free culture medium for hematopoietic stem cell (HSC) expansion, comprising:
a serum-free base medium;
cytokines comprising stem cell factor, thrombopoietin and hematopoietic growth factor Fms-related tyrosine kinase 3 ligand;
an umbilical cord mesenchymal stem cell conditioned medium, derived from culturing human umbilical cord mesenchymal stem cells; and
supplemental components comprising vitamin C, vitamin E or a combination of vitamin C and vitamin E.

2. The serum-free culture medium according to claim 1, wherein the supplemental components include vitamin C.

3. The serum-free culture medium according to claim 1, wherein the supplemental components include vitamin E.

4. The serum-free culture medium according to claim 1, wherein the supplemental components comprise vitamin C and vitamin E.

5. The serum-free culture medium according to claim 4, wherein the supplemental components further comprise estradiol.

6. The serum-free culture medium according to claim 1, wherein the umbilical cord mesenchymal stern cell conditioned medium is produced by a method comprising the following steps:
(a) culturing human umbilical cord mesenchymal stem cells in a cell culture medium;
(b) isolating the cell culture medium by centrifuging the cell culture medium then collecting a supernatant to obtain a conditioned cell culture medium.

7. The serum-free culture medium according to claim 6, the method further comprising step (c): concentrating the conditioned cell culture medium with a 5-10 kilodaltons cut-off membrane to obtain a concentrated umbilical cord mesenchymal stem cell conditioned medium.

8. The serum-free culture medium according to claim 7, wherein in the step (c) the umbilical cord mesenchymal stem cell conditioned medium is 7 to 12 times concentrated by volume.

9. The serum-free culture medium according to claim 8, wherein the umbilical cord mesenchymal stem cell conditioned medium is concentrated to a protein concentration of 50-200 mg/ml.

10. The serum-free culture medium according to claim 1, wherein protein components within the umbilical cord mesenchymal stem cell conditioned medium have a molecular weight of more than 5 kilodaltons.

11. The serum-free culture medium according to claim 1, wherein the cytokines further comprise interleukin 3 and interleukin 6.

12. The serum-free culture medium according to claim 1, wherein the cytokines further comprise granulocyte colony stimulating factor.

13. The serum-free culture medium according to claim 1, wherein the umbilical cord mesenchymal stem cell conditioned medium comprises at least one HSC expansion related proteins selected from the following group: secreted protein acidic and rich in cysteine (SPARC), Follistatin-related protein 1, Metalloproteinase inhibitor 1, Macrophage colony-stimulating factor 1 receptor, Periostin, Galectin-1, CD166 antigen, Far upstream element-binding protein 1, or any combination thereof.

14. A method for expanding hematopoietic stem cells, comprising the following steps:
preparing a serum-free culture medium, the serum-free culture medium is prepared by mixing a serum-free base medium with cytokines, an umbilical cord mesenchymal stem cell conditioned medium and supplemental components, wherein the cytokines comprises stem cell factor, thrombopoietin and hematopoietic growth factor Flt3 ligand, the umbilical cord mesenchymal stem cell conditioned medium is derived from culturing human umbilical cord mesenchymal stem cells, and the supplemental components comprises vitamin C, vitamin E or a combination of vitamin C and vitamin E; and
culturing hematopoietic stem cells in the serum-free culture medium for a first duration.

15. The method for expanding hematopoietic stem cells according to claim 13, further comprising replenishing 50-80% of the serum-free culture medium after the first duration and continuing to culture for a second duration.

16. The method for expanding hematopoietic stem cells according to claim 15, wherein the first duration and the second duration are in the range of 1-20 days.

17. The method for expanding hematopoietic stem cells according to claim 14, wherein the umbilical cord mesenchymal stern cell conditioned medium is produced by a method comprising the following steps:
(a) culturing human umbilical cord mesenchymal stem cells in a cell culture medium;
(b) isolating the cell culture medium by centrifuging the cell culture medium then collecting a supernatant to obtain a conditioned cell culture medium.

18. The method for expanding hematopoietic stem cells according to claim 17, further comprising step (c): concentrating the conditioned cell culture medium with a 5-10 kilodaltons cut-off membrane to obtain a concentrated umbilical cord mesenchymal stem cell conditioned medium.
